SEQUENCE LISTING (2 sequences)

SEQ ID NO: 1 - DNA, 24 bp, Mus musculus
ctccgggttacttcggggcataca

SEQ ID NO: 2 - DNA, 24 bp, Homo sapiens
ccccgggttactttggggcattgc

(19) United States
(12) Patent Application Publication
(10) Pub. No.: US 2005/0036992 A1
(43) Pub. Date: Feb. 17, 2005

(54) Novel use of liver X receptor agonists

(75) Inventors: Enrique Saez, San Diego, CA (US); Peter Tontonoz, Sherman Oaks, CA (US); Bryan A. Laffitte, Raleigh, NC (US); Jing Li, La Jolla, CA (US)

Correspondence Address: GENOMICS INSTITUTE OF THE;NOVARTIS RESEARCH FOUNDATION, 10675 JOHN JAY HOPKINS DRIVE, SUITE E225, SAN DIEGO, CA, 92121-1127, US

(73) Assignees: IRM LLC, Hamilton (BM); The Regents of the University of California, Oakland, CA (US)

(21) Appl. No.: 10/745,334

(22) Filed: Dec. 22, 2003

Related U.S. Application Data
(60) Provisional application No. 60/436,112, filed on Dec. 23, 2002.

(51) Int. Cl. A61K 48/00; C12N 5/08; C12N 15/85

(52) U.S. Cl. 424/93.21; 435/455; 435/366

(57) ABSTRACT

This invention provides novel methods for modulating expression of glut4 and other genes involved in glucose metabolism, and methods for treating or ameliorating diabetes and related diseases. The methods comprise administering to cells in a subject an effective amount of an LXR agonist and thereby modulating expression of those genes that are important for glucose uptake or gluconeogenesis. The modulation will lead to increased uptake of glucose by cells in the subject and/or reduced glucose output in the liver, and accordingly ameliorate symptoms associated with, e.g., type II diabetes.

### CROSS-REFERENCE TO RELATED APPLICATIONS

This application claims the benefit of priority to U.S. Provisional Patent Application Ser. No. 60/436,112 (filed Dec. 23, 2002), the disclosure of which is incorporated herein by reference in its entirety and for all purposes.

### FIELD OF THE INVENTION

The present invention generally relates to methods for modulating expression of glucose metabolism-related genes and to methods for treating diabetes mellitus and related disorders. More particularly, the invention pertains to use of LXR agonists to modulate expression of glut4 and other glucose metabolism-related genes, and to enhance glucose uptake and/or reduce gluconeogenesis in subjects suffering from type II diabetes.

### BACKGROUND OF THE INVENTION

Type II or noninsulin-dependent diabetes mellitus (NIDDM) is a polygenic disease and accounts for >90% of diabetes cases. This disease is characterized by resistance to insulin action on glucose uptake and impaired insulin action to inhibit hepatic glucose production.

Regulation of glucose metabolism by insulin is a key mechanism by which homeostasis is maintained in an animal. Insulin stimulates uptake of glucose from the blood into tissues, especially muscle and fat. This occurs via increased translocation of Glut4, the insulin-sensitive glucose transporter, from an intracellular vesicular compartment to the plasma membrane. Glut4 is the most important insulin-sensitive glucose transporter in these tissues. Insulin binds to its receptor in the plasma membrane, generating a series of signals that result in the translocation or movement of Glut4 transporter vesicles to the plasma membrane.

Liver X receptors (LXRs) are members of a nuclear receptor superfamily that induce ligand dependent transcriptional activation of target genes. They play important roles in cholesterol metabolism and homeostasis (see Janowski, et al., Nature 383:728-731, 1996; and Alberti et al., J Clin Invest 107: 565-73, 2001). Two LXR proteins (α and β) are known to exist in mammals. The expression of LXRα is restricted, with the highest levels being found in the liver, and lower levels found in kidney, intestine, spleen, and adrenals (Willy et al., Genes Dev. 9: 1033-45, 1995). LXRβ is rather ubiquitous, being found in nearly all tissues examined. LXRα and LXRβ are closely related and share 77% amino acid identity in both their DNA- and ligand-binding domains. The LXRs are also conserved between humans and other animals (e.g., rodents).

Like other nuclear receptors, LXRs heterodimerize with retinoid X receptor (RXR) for function. LXRs are known to be activated by certain naturally occurring, oxidized derivatives of cholesterol, including 22(R)-hydroxycholesterol, 24(S)-hydroxycholesterol and 24,25(S)-epoxycholesterol (see Lehmann et al., J. Biol. Chem. 272: 3137-3140, 1997).

### SUMMARY OF THE INVENTION

The present invention provides methods for enhancing glut4 expression in a cell. The methods entail (i) providing a cell expressing glut4 gene, and (ii) administering to the cell an LXR agonist. Some of the methods are directed to enhancing glut4 expression in adipose cells. In some of the methods, the LXR agonist employed is an LXRβ agonist, e.g., GW3965, F3MethylAA, or T0901317. Some of the methods can further comprise measuring glut4 expression level in the cell before and/or after administering the LXR agonist.

In some of the methods, the cell is present in a subject (e.g., a mammal). In some these methods, the subject is suffering from type II diabetes. In these methods, the subject can be administered with a pharmaceutical composition comprising an effective amount of the LXR agonist. Optionally, the subject is administered simultaneously with a known anti-diabetic drug to the subject, e.g., metformin.

In a related aspect, the invention provides methods for enhancing glut4 expression level in a cell. The methods comprise (i) screening test agents to identify an LXR agonist, and (ii) administering to the cell an effective amount of the LXR agonist; thereby enhancing glut4 expression level in the cell. In some of these methods, the cell is an adipose cell. In some methods, the cell is present in a subject (e.g., a mammal). In some of these methods, the subject is suffering from type II diabetes. In some methods, the LXR agonist is an LXRβ agonist.

In another aspect, the present invention provides methods for ameliorating type II diabetes in a subject. These methods entail (i) screening test agents to identify an LXR agonist, and (ii) administering to the subject an effective amount of the LXR agonist; thereby ameliorating type II diabetes in the subject. Additionally, the methods can include measuring circulating glucose level in the subject before and/or after administering the LXR agonist. In some of these methods, the LXR agonist employed is an LXRβ agonist. In some methods, the LXR agonist is administered to the subject at least daily for at least 14 days. Some of the methods can further comprise administering to the subject a known anti-diabetic drug.

In one aspect, the invention provides methods for enhancing insulin sensitivity and glucose uptake by a cell in a subject. The methods comprise administering to the subject an effective amount of an LXR agonist; thereby enhancing insulin sensitivity and glucose uptake by the cell. In some of these methods, the subject is suffering from type II diabetes. In some of the methods, the LXR agonist employed is an LXRβ agonist, e.g., GW3965. Some of the methods are directed to adipose cells in the subject.

In another related aspect, the invention provides methods for reducing gluconeogenesis in a subject. The methods entail (i) screening test agents to identify an LXR agonist, and (ii) administering to the subject an effective amount of the LXR agonist; thereby reducing gluconeogenesis in the subject. Some of the methods are directed to subjects who are suffering from type II diabetes. In some methods, the LXR agonist employed is an LXRβ agonist, e.g., GW3965. The LXR agonist employed in these methods can enhance expression of glucokinase gene, or inhibit expression of at least one of several other gluconeogenesis-related genes (e.g., PGC-1, PEPCK, or glucose-6-phosphatase) in liver of the subject.

A further understanding of the nature and advantages of the present invention may be realized by reference to the remaining portions of the specification and claims.

### BRIEF DESCRIPTION OF THE DRAWINGS

FIGS. 1A-1B show coordinated regulation of genes involved in glucose metabolism by LXR agonist in liver and white adipose tissue.

FIGS. 2A-2B show activity of LXR ligands on gene expression in skeletal muscle and white adipose.

FIGS. 3A-3B show that LXR agonist effects on expression of glucose metabolism-related genes are dependent on LXR expression.

FIG. 4 shows that fasting does not alter expression of LXRs.

FIGS. 5A-5B show that LXR agonists regulate PGC-1 and GLUT4 expression in a cell autonomous manner.

FIGS. 6A-6B show modulation of Glut4 expression in macrophages by LXR ligands.

FIG. 7 shows sequence alignment of LXREs in the mouse and human GLUT4 promoters (SEQ ID NOs: 1 and 2).

FIGS. 8A-8B show that the GLUT4 promoter is a direct target for regulation by LXR/RXR heterodimers.

FIG. 9 shows that an LXR ligand promotes glucose uptake in 3T3-L1 adipocytes.

FIGS. 10A-10B show that an LXR ligand improves glucose tolerance in a model of diet-induced obesity and insulin resistance.

FIG. 11 shows a synergistic effect between an LXR ligand and a known anti-diabetic drug in reducing circulating glucose level.

### DETAILED DESCRIPTION

I. Overview

The present invention is predicated in part on the unexpected discovery that LXR agonists improve glucose tolerance and enhance glut4 expression. The present inventors discovered that there is a coordinate regulation of genes involved in glucose metabolism in liver and adipose tissue. In the liver, LXR agonists inhibit expression of several genes that are important for hepatic gluconeogenesis, e.g., PGC-1, phosphoenolpyruvate carboxykinase (PEPCK), and glucose-6-phosphatase expression. Inhibition of these gluconeogenic genes was accompanied by an induction in expression of glucokinase which promotes hepatic glucose utilization. It was also found that glut4 mRNA levels were upregulated by LXR agonists in adipose tissue, and that glucose uptake in 3T3-L1 adipocytes was enhanced in vitro (see Examples below).

In accordance with these discoveries, the present invention provides methods for enhancing glut4 expression in cells in a subject by administering an LXR agonist to the subject. The LXR agonist can be any of the LXR agonists known in the art. Alternatively, novel LXR agonists can be screened for administering to the subject. The present invention also provides methods for treating diabetes mellitus and related disorders, such as obesity or hyperglycemia, by administering to a subject an effective amount of an LXR agonist to ameliorate the symptoms of the disease. For example, type II diabetes is amenable to treatment with methods of the present invention. By enhancing sensitivity to insulin and glucose uptake by cells, administration with an LXR agonist can also treat other diseases characterized by insulin dysfunction (e.g., resistance, inactivity or deficiency) and/or insufficient glucose transport into cells.

As noted above, the present inventors found that LXR agonists regulate expression levels of a number of genes that play important roles in liver gluconeogenesis. Accordingly, the present invention further provides methods for reducing gluconeogenesis in a subject by modulating expression of such genes (e.g., PGC-1 and PEPCK). These methods comprise (i) screening test agents to identify an LXR agonist and (ii) administering to the subject an effective amount of the LXR agonist. The methods may further comprise detecting a modulatory effect of the LXR agonist on expression of one of these genes in a liver cell of the subject.

The following sections provide guidance for making and using the compositions of the invention, and for carrying out the methods of the invention.

II. Definitions

Unless defined otherwise, all technical and scientific terms used herein have the same meaning as commonly understood by those of ordinary skill in the art to which this invention pertains. The following references provide one of skill with a general definition of many of the terms used in this invention: Singleton et al., DICTIONARY OF MICROBIOLOGY AND MOLECULAR BIOLOGY (2d ed. 1994); THE CAMBRIDGE DICTIONARY OF SCIENCE AND TECHNOLOGY (Walker ed., 1988); and Hale & Marham, THE HARPER COLLINS DICTIONARY OF BIOLOGY (1991). In addition, the following definitions are provided to assist the reader in the practice of the invention.

The term “agent” or “test agent” includes any substance, molecule, element, compound, entity, or a combination thereof. It includes, but is not limited to, e.g., protein, polypeptide, small organic molecule, polysaccharide, polynucleotide, and the like. It can be a natural product, a synthetic compound, or a chemical compound, or a combination of two or more substances. Unless otherwise specified, the terms “agent”, “substance”, and “compound” can be used interchangeably.

The term “analog” is used herein to refer to a molecule that structurally resembles a reference molecule but which has been modified in a targeted and controlled manner, by replacing a specific substituent of the reference molecule with an alternate substituent. Compared to the reference molecule, an analog would be expected, by one skilled in the art, to exhibit the same, similar, or improved utility. Synthesis and screening of analogs, to identify variants of known compounds having improved traits (such as higher binding affinity for a target molecule) is an approach that is well known in pharmaceutical chemistry.

As used herein, “contacting” has its normal meaning and refers to combining two or more agents (e.g., polypeptides or small molecule compounds) or combining agents and cells. Contacting can occur in vitro, e.g., combining two or more agents or combining a test agent and a cell or a cell lysate in a test tube or other container. Contacting can also occur in a cell or in situ, e.g., contacting two polypeptides in a cell by coexpression in the cell of recombinant polynucleotides encoding the two polypeptides, or in a cell lysate.

A “heterologous sequence” or a “heterologous nucleic acid,” as used herein, is one that originates from a source foreign to the particular host cell, or, if from the same source, is modified from its original form. Thus, a heterologous gene in a host cell includes a gene that, although being endogenous to the particular host cell, has been modified. Modification of the heterologous sequence can occur, e.g., by treating the DNA with a restriction enzyme to generate a DNA fragment that is capable of being operably linked to the promoter. Techniques such as site-directed mutagenesis are also useful for modifying a heterologous nucleic acid.

The term “homologous” when referring to proteins and/or protein sequences indicates that they are derived, naturally or artificially, from a common ancestral protein or protein sequence. Similarly, nucleic acids and/or nucleic acid sequences are homologous when they are derived, naturally or artificially, from a common ancestral nucleic acid or nucleic acid sequence. Homology is generally inferred from sequence similarity between two or more nucleic acids or proteins (or sequences thereof). The precise percentage of similarity between sequences that is useful in establishing homology varies with the nucleic acid and protein at issue, but as little as 25% sequence similarity is routinely used to establish homology. Higher levels of sequence similarity, e.g., 30%, 40%, 50%, 60%, 70%, 80%, 90%, 95% or 99% or more can also be used to establish homology. Methods for determining sequence similarity percentages (e.g., BLASTP and BLASTN using default parameters) are described herein.

A “host cell,” as used herein, refers to a prokaryotic or eukaryotic cell into which a heterologous polynucleotide can be or has been introduced. The heterologous polynucleotide can be introduced into the cell by any means, e.g., electroporation, calcium phosphate precipitation, microinjection, transformation, viral infection, and/or the like.

The term “identical”, “sequence identical” or “sequence identity” in the context of two nucleic acid sequences or amino acid sequences refers to the residues in the two sequences which are the same when aligned for maximum correspondence over a specified comparison window. A “comparison window”, as used herein, refers to a segment of at least about 20 contiguous positions, usually about 50 to about 200, more usually about 100 to about 150 in which a sequence may be compared to a reference sequence of the same number of contiguous positions after the two sequences are aligned optimally. Methods of alignment of sequences for comparison are well-known in the art. Optimal alignment of sequences for comparison may be conducted by the local homology algorithm of Smith and Waterman (1981) Adv. Appl. Math. 2:482; by the alignment algorithm of Needleman and Wunsch (1970) J. Mol. Biol. 48:443; by the search for similarity method of Pearson and Lipman (1988) Proc. Nat. Acad. Sci U.S.A. 85:2444; by computerized implementations of these algorithms (including, but not limited to CLUSTAL in the PC/Gene program by Intelligentics, Mountain View, Calif.; and GAP, BESTFIT, BLAST, FASTA, or TFASTA in the Wisconsin Genetics Software Package, Genetics Computer Group (GCG), 575 Science Dr., Madison, Wis., U.S.A.). The CLUSTAL program is well described by Higgins and Sharp (1988) Gene 73:237-244; Higgins and Sharp (1989) CABIOS 5:151-153; Corpet et al. (1988) Nucleic Acids Res. 16:10881-10890; Huang et al (1992) Computer Applications in the Biosciences 8:155-165; and Pearson et al. (1994) Methods in Molecular Biology 24:307-331. Alignment is also often performed by inspection and manual alignment.

The term “substantially identical” nucleic acid or amino acid sequence means that a nucleic acid or amino acid sequence comprises a sequence that has at least 90% sequence identity or more, preferably at least 95%, more preferably at least 98% and most preferably at least 99%, compared to a reference sequence using any of the programs described in the art (preferably BLAST) using standard parameters. For example, the BLASTN program (for nucleotide sequences) uses as defaults a wordlength (W) of 11, an expectation (E) of 10, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff, Proc. Natl. Acad. Sci. USA 89:10915 (1989)). Percentage of sequence identity is determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. Preferably, the substantial identity exists over a region of the sequences that is at least about 50 residues in length, more preferably over a region of at least about 100 residues, and most preferably the sequences are substantially identical over at least about 150 residues. In a most preferred embodiment, the sequences are substantially identical over the entire length of the coding regions.

The term “LXR” (liver X receptor) or “LXR receptor” includes all subtypes of this receptor. Specifically LXR includes LXRα and LXRβ. LXRα has been referred to under a variety of names such as LXRU, LXRa, LXR, RLD-1, NR1H3. It encompasses any polypeptide encoded by a gene with substantial sequence identity to GenBank accession number U22662. Similarly, LXRβ included any polypeptide encoded by a gene referred to as LXRb, LXRP, LXRβ, NER, NER1, UR, OR-1, RIP 15, NR1H2 or a gene with substantial sequence identity to GenBank accession number U07132.

The term “ligand” refers to an agonist or partial agonist of LXR. The ligand may be selective for LXRα or LXRβ, or it may have mixed binding affinity for both LXRa and LXRβ. While a ligand can either agonize or antagonize a receptor function, unless otherwise specified, an LXR ligand used herein primarily refers to an LXR agonist that activated the LXR receptor activities.

The term “nucleic acid” or “polynucleotide” refers to a deoxyribonucleotide or ribonucleotide polymer in either single- or double-stranded form, and unless otherwise limited, encompasses known analogues of natural nucleotides that hybridize to nucleic acids in manner similar to naturally occurring nucleotides.

The term “modulate” with respect to an LXR receptor refers to activation of the LXR receptor and its biological activities associated with the LXR pathway (e.g., transcription regulation of a target gene). Modulation of LXR receptor can be up-regulation (i.e., agonizing, activation or stimulation) or down-regulation (i.e. antagonizing, inhibition or suppression). The mode of action of an LXR modulator can be direct, e.g., through binding to the LXR receptor as a ligand. The modulation can also be indirect, e.g., through binding to and/or modifying another molecule which otherwise binds to and activates the LXR receptor. Thus, modulation of LXR includes a change in the bioactivities of an LXR agonist ligand (i.e., its activity in binding to and/or activating an LXR receptor) or a change in the cellular level of the ligand.

The term “oligonucleotide” refers to an oligomer or polymer of ribonucleic acid or deoxyribonucleic acid. This term includes oligonucleotides composed of naturally-occurring nucleobases, sugars and covalent intersugar (backbone) linkages as well as oligonucleotides having non-naturally-occurring portions which function similarly. Such modified or substituted oligonucleotides are often preferred over native forms because of desirable properties such as, for example, enhanced cellular uptake, enhanced binding to target and increased stability in the presence of nucleases.

The term “operably linked” refers to a functional relationship between two or more polynucleotide (e.g., DNA) segments. Typically, it refers to the functional relationship of a transcriptional regulatory sequence to a transcribed sequence. For example, a promoter or enhancer sequence is operably linked to a coding sequence if it stimulates or modulates the transcription of the coding sequence in an appropriate host cell or other expression system. Generally, promoter transcriptional regulatory sequences that are operably linked to a transcribed sequence are physically contiguous to the transcribed sequence, i.e., they are cis-acting. However, some transcriptional regulatory sequences, such as enhancers, need not be physically contiguous or located in close proximity to the coding sequences whose transcription they enhance. A polylinker provides a convenient location for inserting coding sequences so the genes are operably linked to a promoter. Polylinkers are polynucleotide sequences that comprise a series of three or more closely spaced restriction endonuclease recognition sequences.

The term “polypeptide” is used interchangeably herein with the terms “polypeptides” and “protein(s)”, and refers to a polymer of amino acid residues, e.g., as typically found in proteins in nature. A “mature protein” is a protein which is full-length and which, optionally, includes glycosylation or other modifications typical for the protein in a given cell membrane.

As used herein, the phrase “screening for LXR agonists” refers to use of an appropriate assay system to identify novel LXR agonists from test agents. The assay can be an in vitro or an in vivo assay suitable for identifying whether a test agent can stimulate or activate one or more of the biological functions of the LXR receptor. Examples of suitable bioassays include, but are not limited to, assays for examining binding of test agents to an LXR polypeptide (e.g., LXR fragment containing its ligand binding domain), transcription-based assays, creatine kinase assays, assays based on the differentiation of pre-adipocytes, assays based on glucose uptake control in adipocytes, and immunological assays.

The term “subject” includes mammals, especially humans. It also encompasses other non-human animals that are amenable for treatment with LXR agonists of the present invention.

A “variant” of a molecule such as an LXR receptor or an LXR agonist is meant to refer to a molecule substantially similar in structure and biological activity to either the entire molecule, or to a fragment thereof. Thus, provided that two molecules possess a similar activity, they are considered variants as that term is used herein even if the composition or secondary, tertiary, or quaternary structure of one of the molecules is not identical to that found in the other, or if the sequence of amino acid residues is not identical.

A “vector” is a composition for facilitating introduction, replication and/or expression of a selected nucleic acid in a cell. Vectors include, e.g., plasmids, cosmids, viruses, YACs, bacteria, poly-lysine, etc. A “vector nucleic acid” is a nucleic acid molecule into which heterologous nucleic acid is optionally inserted which can then be introduced into an appropriate host cell. Vectors preferably have one or more origins of replication, and one or more sites into which the recombinant DNA can be inserted. Vectors often have convenient means by which cells with vectors can be selected from those without, e.g., they encode drug resistance genes. Common vectors include plasmids, viral genomes, and (primarily in yeast and bacteria) “artificial chromosomes.” “Expression vectors” are vectors that comprise elements that provide for or facilitate transcription of nucleic acids that are cloned into the vectors. Such elements can include, e.g., promoters and/or enhancers operably coupled to a nucleic acid of interest.

III. LXR Agonists for Modulating Glut4 Expression

There are many LXR agonists that are suitable for practicing methods of the present invention. They can be known agents that activate LXR receptor, e.g., GW3965 (see Examples below), or other commercially available compounds such as F3MethylAA (from Merck; see Menke et al., Endocrinology 143: 2548-58, 2002) and T0901317 (Tularik, Calif.; see Examples below). They can also be novel LXR agonists to be screened for in accordance with the present invention. As detailed below, the LXR agonists suitable for the present invention can be polypeptides, peptides, small molecules, or other agents. The LXR agonists can be agonists for LXR of human as well as other animals.

A great number of LXR agonists have been described in the art. Examples of small molecule LXR agonists include the well known oxysterols and related compounds (Janowski et al., Nature 383: 728-31, 1996); T0901317 and T0314407 (Schultz et al., Genes Dev 14: 2831-8, 2000); 24(S)-hydroxycholesterol, and 22(R)-hydroxycholesterol (Janowski et al., Nature 383: 728-731, 1996); and 24,25-epoxycholesterol (U.S. Pat. No. 6,316,503). Exemplary polypeptide agonists of LXR have also been disclosed in the art, e.g., WO 02/077229. Additional LXR agonists have been described in the art, e.g., in U.S. Pat. No. 6,316,503; Collins et al., J Med Chem. 45: 1963-6, 2002; Joseph et al., Proc Natl Acad Sci USA 99: 7604-9, 2002; Menke et al., Endocrinology 143: 2548-58, 2002; Schultz et al., Genes Dev. 14: 2831-8, 2000; and Schmidt et al., Mol Cell Endocrinol. 155: 51-60, 1999.

Many LXR agonists are effective in activating both LXRα and LXRβ (e.g., GW3965 as described in Collins et al., J Med Chem. 45: 1963-6, 2002). Some LXR agonists activate LXRα and LXRβ under different conditions. For example, 6-alpha-hydroxylated bile acids are agonists of LXRα, but also activate LXRβ at higher concentrations (Song et al., Steroids 65: 423-7, 2000). Some LXR agonists act exclusively on LXRα, while some others activate only LXRβ. For example, introduction of an oxygen on the sterol B-ring of oxysterol results in a ligand with LXRα-subtype selectivity (Janowski et al., Proc Natl Acad Sci USA 96: 266-71, 1999). Using ligand-dependent transcription assays, it was found that 5-tetradecyloxy-2-furancarboxylic acid (TOFA) and hydroxycholesterol transactivates chimeric receptors composed of the glucocorticoid receptor DNA binding domain and the ligand binding regions of LXRβ, PPARα, and PPARβ receptors (Schmidt et al., Mol Cell Endocrinol. 155: 51-60, 1999).

LXR agonists can also be obtained from derivatives of known polypeptide agonists of the LXR receptor. They can be produced by a variety of art known techniques. For example, specific oligopeptides (e.g., 10-25 amino acid residues) spanning a known polypeptide agonist of LXR can be synthesized (e.g., chemically or recombinantly) and tested for their ability to activate an LXR receptor. The LXR agonist fragments can be synthesized using standard techniques such as those described in Bodansky, M. Principles of Peptide Synthesis, Springer Verlag, Berlin (1993) and Grant, G. A (ed.). Synthetic Peptides: A User's Guide, W. H. Freeman and Company, New York (1992). Automated peptide synthesizers are commercially available, e.g., from Advanced ChemTech Model 396; Milligen/Biosearch 9600. Alternatively, such LXR agonists can be produced by digestion of native or recombinantly produced polypeptide agonists of LXR using a protease, e.g., trypsin, thermolysin, chymotrypsin, or pepsin. Computer analysis (using commercially available software, e.g. MacVector, Omega, PCGene, Molecular Simulation, Inc.) can be used to identify proteolytic cleavage sites.

The polypeptide or peptide agonists for use in methods of the present invention are preferably isolated and substantially free of cellular material or other contaminating proteins from the cell or tissue source from which the LXR agonists is derived, or substantially free from chemical precursors or other chemicals when chemically synthesized. The proteolytic or synthetic polypeptide agonists or their fragments can comprise as many amino acid residues as are necessary to activate LXR receptor activity, and can comprise at least 5, 10, 15, 20, 25, 30, 35, 40, 45, 50, or more amino acids in length.

IV. Screening for Novel LXR Agonists

A. General Scheme

Other than known compounds and polypeptides that activate the LXR receptor, LXR agonists can also be obtained by screening test agents (e.g., compound libraries) to identify novel LXR agonists that bind to and/or activate LXR receptor activities. To screen for such novel LXR agonists, a human LXR or LXR of other animals can be employed in a proper assay system. Polynucleotide and amino acid sequences of the LXR receptors are known and described in the art. Their structures and functional organizations, including their ligand binding domains, have also been characterized. See, e.g., Apfel et al., Mol Cell Biol 14: 7025-7035, 1994; Willy et al., Genes Dev 9: 1033-1045, 1995; Song et al., Proc Natl Acad Sci USA 91: 10809-10813, 1994; Shinar et al., Gene 147: 273-276, 1994; Teboul et al., Proc Natl Acad Sci USA 92: 2096-2100, 1995; and Seol et al., Mol Endocrinol 9: 72-85, 1995.

The agonists can activate either LXR or LXRβ. In addition, instead of the full length LXR molecule, some of the screen assays can employ an LXR polypeptide that comprises a fragment of an LXR molecule. For example, the two functional domains of the LXR receptor, the N-terminal DNA binding domain (DBD) and the C-terminal ligand-binding domain (LBD), mediate the transcriptional activation function of nuclear receptors. An LXR polypeptide containing any of these domains can be used in screening for novel LXR agonists.

A number of assay systems can be employed to screen test agents for agonists of an LXR receptor. As detailed below, test agents can be screened for direct binding to an LXR polypeptide or a fragment thereof (e.g., its ligand binding domain). Alternatively or additionally, potential LXR agonists can be examined for ability to activate LXR receptor pathway or stimulate other biological activities of the LXR receptor. Either an in vitro assay system or a cell-based assay system can be used in the screening.

Selectivity of potential LXR agonists for different receptors (e.g., LXRα, LXRβ, RXR, or PPAR) can be tested using methods well known in the art, e.g., the LXR radioligand competition scintillation proximity assays described in, e.g., WO 01/41704, and the PPAR competition binding assays described in, e.g., Berger et al., J Biol Chem 274: 6718-6725, 1999).

B. Test Agents

Test agents that can be screened for novel LXR agonists include polypeptides, beta-turn mimetics, polysaccharides, phospholipids, hormones, prostaglandins, steroids, aromatic compounds, heterocyclic compounds, benzodiazepines, oligomeric N-substituted glycines, oligocarbamates, polypeptides, saccharides, fatty acids, steroids, purines, pyrimidines, derivatives, structural analogs or combinations thereof. Some test agents are synthetic molecules, and others natural molecules.

Test agents are obtained from a wide variety of sources including libraries of synthetic or natural compounds. Combinatorial libraries can be produced for many types of compound that can be synthesized in a step-by-step fashion. Large combinatorial libraries of compounds can be constructed by the encoded synthetic libraries (ESL) method described in WO 95/12608, WO 93/06121, WO 94/08051, WO 95/35503 and WO 95/30642. Peptide libraries can also be generated by phage display methods (see, e.g., Devlin, WO 91/18980). Libraries of natural compounds in the form of bacterial, fungal, plant and animal extracts can be obtained from commercial sources or collected in the field. Known pharmacological agents can be subject to directed or random chemical modifications, such as acylation, alkylation, esterification, amidification to produce structural analogs.

Combinatorial libraries of peptides or other compounds can be fully randomized, with no sequence preferences or constants at any position. Alternatively, the library can be biased, i.e., some positions within the sequence are either held constant, or are selected from a limited number of possibilities. For example, in some cases, the nucleotides or amino acid residues are randomized within a defined class, for example, of hydrophobic amino acids, hydrophilic residues, sterically biased (either small or large) residues, towards the creation of cysteines, for cross-linking, prolines for SH-3 domains, serines, threonines, tyrosines or histidines for phosphorylation sites, or to purines.

The test agents can be naturally occurring proteins or their fragments. Such test agents can be obtained from a natural source, e.g., a cell or tissue lysate. Libraries of polypeptide agents can also be prepared, e.g., from a cDNA library commercially available or generated with routine methods. The test agents can also be peptides, e.g., peptides of from about 5 to about 30 amino acids, with from about 5 to about 20 amino acids being preferred, and from about 7 to about 15 being particularly preferred. The peptides can be digests of naturally occurring proteins, random peptides, or “biased” random peptides. In some methods, the test agents are polypeptides or proteins.

The test agents can also be nucleic acids. Nucleic acid test agents can be naturally occurring nucleic acids, random nucleic acids, or “biased” random nucleic acids. For example, digests of prokaryotic or eukaryotic genomes can be similarly used as described above for proteins.

In some preferred methods, the test agents are small organic molecules (e.g., molecules with a molecular weight of not more than about 1,000). Preferably, high throughput assays are adapted and used to screen for such small molecules. In some methods, combinatorial libraries of small molecule test agents as described above can be readily employed to screen for small molecule modulators of an LXR receptor. A number of assays are available for such screening, e.g., as described in Schultz (1998) Bioorg Med Chem Lett 8:2409-2414; Weller (1997) Mol Divers. 3:61-70; Fernandes (1998) Curr Opin Chem Biol 2:597-603; and Sittampalam (1997) Curr Opin Chem Biol 1:384-91.

Potential LXR agonists can also be identified based on rational design. For example, Janowski et al. (Proc Natl Acad Sci USA 96: 266-71, 1999) disclosed structural requirements of ligands for LXRα and LXRβ. It was shown that position-specific monooxidation of the sterol side chain of oxysterol is requisite for LXR high-affinity binding and activation. Enhanced binding and activation can also be achieved through the use of 24-oxo ligands that act as hydrogen bond acceptors in the side chain. In addition, introduction of an oxygen on the sterol B-ring results in a ligand with LXRα-subtype selectivity.

Libraries of test agents to be screened with the claimed methods can also be generated based on structural studies of the LXR receptors, their fragments or analogs. Such structural studies allow the identification of test agents that are more likely to bind to the LXR receptor. The three-dimensional structure of an LXR receptor can be studied in a number of ways, e.g., crystal structure and molecular modeling. Methods of studying protein structures using x-ray crystallography are well known in the literature. See Physical Bio-chemistry, Van Holde, K. E. (Prentice-Hall, N.J. 1971), pp. 221-239, and Physical Chemistry with Applications to the Life Sciences, D. Eisenberg & D. C. Crothers (Benjamin Cummings, Menlo Park 1979). Methods of molecular modeling have been described in the literature, e.g., U.S. Pat. No. 5,612,894 entitled “System and method for molecular modeling utilizing a sensitivity factor”, and U.S. Pat. No. 5,583,973 entitled “Molecular modeling method and system”. In addition, protein structures can also be determined by neutron diffraction and nuclear magnetic resonance (NMR). See, e.g., Physical Chemistry, 4th Ed. Moore, W. J. (Prentice-Hall, N.J. 1972), and NMR of Proteins and Nucleic Acids, K. Wuthrich (Wiley-Interscience, New York 1986).

C. Screening Test Agents for Binding to an LXR Polypeptide

In some screening assays, binding of a test agent to an LXR or an LXR polypeptide containing its ligand binding domain is determined. Binding of test agents (e.g., polypeptides) to the LXR polypeptide can be assayed by a number of methods including, e.g., labeled in vitro protein-protein binding assays, electrophoretic mobility shift assays, immunoassays for protein binding, functional assays (phosphorylation assays, etc.), and the like. See, e.g., U.S. Pat. Nos. 4,366,241; 4,376,110; 4,517,288; and 4,837,168; and also Bevan et al., Trends in Biotechnology 13:115-122, 1995; Ecker et al., Bio/Technology 13:351-360, 1995; and Hodgson, Bio/Technology 10:973-980, 1992. The test agent can be identified by detecting a direct binding to the LXR polypeptide, e.g., co-immunoprecipitation with the LXR polypeptide by an antibody directed to the LXR polypeptide. The test agent can also be identified by detecting a signal that indicates that the agent binds to the LXR polypeptide, e.g., fluorescence quenching.

Competition assays provide a suitable format for identifying test agents (e.g., peptides or small molecule compounds) that specifically bind to an LXR polypeptide. In such formats, test agents are screened in competition with a compound already known to bind to the LXR polypeptide. The known binding compound can be a synthetic compound. It can also be an antibody, which specifically recognizes the LXR polypeptide, e.g., a monoclonal antibody directed against the LXR polypeptide. If the test agent inhibits binding of the compound known to bind the LXR polypeptide, then the test agent also binds the LXR polypeptide.

Numerous types of competitive binding assays are known, for example: solid phase direct or indirect radioimmunoassay (RIA), solid phase direct or indirect enzyme immunoassay (EIA), sandwich competition assay (see Stahli et al., Methods in Enzymology 9:242-253 (1983)); solid phase direct biotin-avidin EIA (see Kirkland et al., J. Immunol. 137:3614-3619 (1986)); solid phase direct labeled assay, solid phase direct labeled sandwich assay (see Harlow and Lane, “Antibodies, A Laboratory Manual,” Cold Spring Harbor Press (1988)); solid phase direct label RIA using ¹²⁵I label (see Morel et al., Mol. Immunol. 25(1):7-15 (1988)); solid phase direct biotin-avidin EIA (Cheung et al., Virology 176:546-552 (1990)); and direct labeled RIA (Moldenhauer et al., Scand. J. Immunol. 32:77-82 (1990)). Typically, such an assay involves the use of purified polypeptide bound to a solid surface or cells bearing either of these, an unlabelled test agent and a labeled reference compound. Competitive inhibition is measured by determining the amount of label bound to the solid surface or cells in the presence of the test agent. Usually the test agent is present in excess. Modulating agents identified by competition assay include agents binding to the same epitope as the reference compound and agents binding to an adjacent epitope sufficiently proximal to the epitope bound by the reference compound for steric hindrance to occur. Usually, when a competing agent is present in excess, it will inhibit specific binding of a reference compound to a common target polypeptide by at least 50 or 75%.

The screening assays can be either in insoluble or soluble formats. One example of the insoluble assays is to immobilize an LXR polypeptide or its fragments onto a solid phase matrix. The solid phase matrix is then put in contact with test agents, for an interval sufficient to allow the test agents to bind. After washing away any unbound material from the solid phase matrix, the presence of the agent bound to the solid phase allows identification of the agent. The methods can further include the step of eluting the bound agent from the solid phase matrix, thereby isolating the agent. Alternatively, other than immobilizing the LXR polypeptide, the test agents are bound to the solid matrix and the LXR polypeptide molecule is then added.

Soluble assays include some of the combinatory libraries screening methods described above. Under the soluble assay formats, neither the test agents nor the LXR polypeptide are bound to a solid support. Binding of an LXR polypeptide or fragment thereof to a test agent can be determined by, e.g., changes in fluorescence of either the LXR polypeptide or the test agents, or both. Fluorescence may be intrinsic or conferred by labeling either component with a fluorophor.

In some binding assays, either the LXR polypeptide, the test agent, or a third molecule (e.g., an antibody against the LXR polypeptide) can be provided as labeled entities, i.e., covalently attached or linked to a detectable label or group, or cross-linkable group, to facilitate identification, detection and quantification of the polypeptide in a given situation. These detectable groups can comprise a detectable polypeptide group, e.g., an assayable enzyme or antibody epitope. Alternatively, the detectable group can be selected from a variety of other detectable groups or labels, such as radiolabels (e.g., ¹²⁵I, ³²P, ³⁵S) or a chemiluminescent or fluorescent group. Similarly, the detectable group can be a substrate, cofactor, inhibitor or affinity ligand.

Binding of a test agent to LXR can also be tested indirectly with a cell-based assay. For example, a DNA-binding domain of the nonreceptor transcription factor GAL4 can be fused to the ligand-binding domain of LXR (e.g., LXRα). The resultant construct is introduced into a host cell (e.g., the 293 cells) together with a reporter construct (e.g., a UAS-containing luciferase reporter construct). The transfected cells are then treated with libraries of test agents, and reporter polypeptide activity (e.g., luciferase activity) is measured. Effects of individual test agents on the reporter polypeptide activity are evaluated relative to a control (i.e., when no test compound is present).

The cell-free ligand sensing assay (LiSA) can also be employed to identify novel LXR agonists. It can be performed as described in the art, e.g., Collins et al., J Med Chem. 45: 1963-6, 2002; and Spencer et al., J. Med. Chem. 44: 886-97, 2001. This assay measures the ligand-dependent recruitment of a peptide from the steroid receptor coactivator 1 (SRC1) to the nuclear receptor. With this assay (LiSA), the structural requirements for activation of the LXR receptor by test agents can be studied.

D. Screening Test Agents for Ability to Modulate LXR Cellular Activities

Other than or in addition to detecting a direct binding of a test agent to an LXR polypeptide, potential LXR agonists for use in the methods of the present invention can also be examined for ability to activate other bioactivities or cellular activities of the LXR receptor. Test agents which activate LXR receptor can be identified by monitoring their effects on a number of LXR cellular activities. LXR cellular activities include any activity mediated by activated LXR receptor (e.g., transcriptional regulation of a target gene). For example, LXR trans-activate expression of a number of target genes (e.g., ABCA1), inhibit fibroblast differentiation to adipocytes, modulate the production of muscle-specific enzymes, e.g., creatine kinase, modulate glucose uptake by cells, and stimulate myoblast cell proliferation. The degree to which a test agent activates an LXR receptor can be identified by testing for the ability of the agent to enhance such LXR activities.

Thus, a novel LXR agonist can be identified by identifying a test agent that enhances expression of an LXR target gene (e.g., ABCA 1, ABCG 1, SREBP 1, or the cholesterol 7-hydroxylase gene). Methods for identifying test agents that induce an LXR target gene expression (e.g., increasing ABCA1 mRNA levels) have been disclosed in the art, e.g., Menke et al., Endocrinology 143: 2548-58, 2002; Sparrow et al., J. Biol. Chem. 277: 10021-7, 2002; and Murthy et al., J Lipid Res. 43: 1054-64, 2002.

Other than monitoring LXR target gene expression, LXR agonists can also be identified by examining other cellular activities stimulated by the LXR pathway. For example, LXR agonists modulate the protein level and hence activity of a muscle-specific enzyme, creatine kinase. Therefore, LXR agonists can be screened by examining test agents for ability to modulate creatine kinase activity, e.g., as described in Somjen et al., J Steroid Biochem Mol Biol 62: 401-8, 1997. The assay can be performed in a cell line, e.g., the mouse skeletal myoblast cell line or a primary chick myoblast cell line. Effects of test compounds on creatine kinase activity in the cultured cells can be measured in the cell lysates using a commercially available kit (available by Sigma, St Louis, Mo.).

Modulation of other cellular bioactivities of the LXR receptor can also be detected using methods well known and routinely practiced in the art. For example, the test agent can be assayed for their activities in increasing cholesterol efflux from cells such as macrophages (Menke et al., Endocrinology 143: 2548-58, 2002; and Sparrow et al., J. Biol. Chem. 277: 10021-7, 2002). Other assays include ligand-dependent transcription assays (Schmidt et al., Mol Cell Endocrinol 155: 51-60, 1999), methods for measuring the ability of LXR agonists to interfere with the differentiation process of pre-adipocytes (fibroblasts) to adipocytes (Plaas et al., Biosci Rep 1: 207-16, 1981; Hiragun et al., J Cell Physiol 134: 124-30, 1988; and Liao et al., J Biol Chem 270: 12123-32, 1995), or the ability to stimulate myoblast cell proliferation (Konishi et al., Biochemistry 28: 8872-7, 1989; and Austin et al., J Neurol Sci 101: 193-7, 1991). As a control, all these assays can include measurements before and after the test agent is added to the assay system.

V. Therapeutic Applications

The present invention provides methods for modulating expression of genes involved in glucose transport (e.g., glut4) and gluconeogenesis (e.g., PGC-1 or PEPCK). These methods of the invention can be used either in vitro or in vivo to increase insulin sensitivity and/or glucose uptake by a cell, as well as reducing glucose output by the liver cells. The methods also find application in treating a disease characterized by insufficient glut4 expression, insulin dysfunction (e.g., resistance, inactivity or deficiency) and/or insufficient glucose transport into cells. Such diseases include, but are not limited to diabetes, hyperglycemia and obesity. Modulation of glut4 expression is also useful for preventing or modulating the development of such diseases or disorders in a subject suspected of being, or known to be, prone to such diseases or disorders. The LXR agonists to be used in these applications can be any of the known LXR agonists that have been described in the art. Alternatively, the therapeutic methods comprise screening test agents to identify novel LXR agonists as described above, and administering such novel agonists to enhance glut4 expression in cells or to treat the above noted diseases in a subject.

A. Modulating Gene Expression In Cells

Methods of the present invention can be used to modulate of expression of a number of genes that are involved in glucose metabolism. For example, the invention provides methods for enhancing glut4 expression in fat cells or muscle cells such as white adipose cells and smooth muscle cells. Similarly, liver expression of gluconeogenesis-related genes can be inhibited or reduced in accordance with methods of the invention. Such genes include PGC-1, PEPCK, glucose-6-phosphatase, and glucokinase. Cells suitable for modulation include isolated cells maintained in culture, as well as cells within their natural context in vivo in a subject, e.g., in the liver, fat tissue or muscle tissue such as pectoralis, triceps, gastrocnemius, quadriceps, and iliocostal muscles of a mammal.

To modulate expression of these genes in vivo, a cell can be contacted with any a number of the known LXR agonists or novel LXR agonists identified in accordance with the present invention. In some methods, an LXR agonist is introduced directly to a subject (e.g., a human or a non-human subject). In some methods, a polynucleotide encoding a polypeptide agonist of an LXR receptor is introduced by retroviral or other means (as detailed below). In some methods, an LXR agonist specific for the LXRa receptor is used. In some methods, an LXRβ receptor-specific agonist is employed. In still some other methods, agonists that can activate both LXRα and LXRβ are administered to cells to modulate the gene expression.

In some methods, the cell is first determined to have low expression level of the relevant gene (e.g., glut4 level in an adipose cell) as compared to normal level (“baseline level,” or “a desired level”) of the same cell type. In some applications, expression levels of the genes are measured before and/or after treatment with the LXR agonist in order to confirm that the treatment results in modulated expression level of the genes. When the LXR agonist is administered to a subject, the in vivo effect can be monitored by taking a tissue sample from the subject and analyzing expression levels of the genes to be modulated, e.g., glut4 in adipose tissue or PGC-1 in liver cells. The tissue or cell samples can be obtained by following the well-established and routinely practiced medical procedures. Animal adipose tissue sample (e.g., needle biopsy from subcutaneous adipose tissue) can be easily obtained as described in, e.g., Martinsson et al., J Med Lab Technol, 24: 52-3, 1967; Novak et al., Exp Cell Res 73: 335-44, 1972; and Taskinen et al., Clin Chim Acta 104: 107-17, 1980. Percutaneous adipose tissue biopsy can be obtained by mini-liposuction method as described in, e.g., Bastard et al., J Parenter Enteral Nutr 18: 466-8, 1994. Similarly, a small liver tissue sample from a subject (e.g., an animal) can be obtained by the well established liver biopsy methods as described in e.g., Oxender et al., J Dairy Sci 54: 286-8, 1971; Spiezia et al., Eur J Ultrasound 15: 127-31, 2002; and Rinella et al., Liver Transpl. 8: 1123-5, 2002.

Activities of LXR agonists in enhancing human glut4 expression or reducing expression of other genes (e.g., PGC-1 or PEPCK) can be examined or further verified in vivo by employing transgenic animals. Accordingly, transgenic animals with integrated human genes (e.g., glut4 or PGC-1) and LXR-encoding sequences can be used to assay induction of glut4 expression in vivo. Transgenic animals (e.g., transgenic mice) harboring the human sequences can be generated according to methods well known in the art. For example, techniques routinely used to create and screen for transgenic animals have been described in, e.g., see Bijvoet (1998) Hum. Mol. Genet. 7:53-62; Moreadith (1997) J. Mol. Med. 75:208-216; Tojo (1995) Cytotechnology 19:161-165; Mudgett (1995) Methods Mol. Biol. 48:167-184; Longo (1997) Transgenic Res. 6:321-328; U.S. Pat. No. 5,616,491 (Mak, et al.); U.S. Pat. Nos. 5,464,764; 5,631,153; 5,487,992; 5,627,059; 5,272,071; and, WO 91/09955, WO 93/09222, WO 96/29411, WO 95/31560, and WO 91/12650.

As noted above, the present invention also encompasses therapeutic methods for treating or ameliorating diabetes mellitus and related disorders such as obesity or hyperglycemia. For example, the connection between glut4 expression and diabetes, especially type II diabetes mellitus is well documented (J Clin Endocrinol Metab 77: 25-6, 1993). Glut4 is primarily expressed in adipose and muscle tissues. It was suggested that a reduction in glut4 expression in slow fibers reduces the insulin-sensitive Glut4 pool in type II diabetes and thus contributes to skeletal muscle insulin resistance (Gaster et al., Diabetes 50: 1324-9, 2001). Muscle-specific inactivation of Glut4 caused glucose toxicity and the development of diabetes in mice (Kim et al., J Clin Invest 108: 153-60, 2001). A compound which improves peripheral insulin resistance in type II diabetic subjects and animal models apparently exerts beneficial effects by increasing glut4 expression in adipose tissue (Furuta et al., Diabetes Res Clin Pract 56: 159-71, 2002). Thus, by enhancing glut4 expression, administration of LXR agonists to a subject suffering from diabetes (e.g., type II diabetes) can lead to therapeutic effects. In some embodiments of the present invention, therapeutical effects are monitored by measuring circulating glucose level in the subject before and/or after administering an LXR agonist. Glucose level in the subject can be measured with methods well known in the art. For example, blood glucose levels can be measured very simply and quickly with many commercially available blood glucose testing kits.

The present inventors observed that modulation of expression of glucose metabolism-related genes can be achieved after application of an LXR agonist for a very short period of time, e.g., in 3 days. However, when the objective is to enhance insulin sensitivity or to ameliorate symptoms of diabetes in a subject, a longer period of treatment is necessary. For such applications, the LXR agonist is typically administered to a subject for a continued period of time, e.g., at least 10 days, 14 days, 30 days, 60 days, 90 days, or longer.

B. Pharmaceutical Compositions

The LXR agonists of the present invention can be directly administered under sterile conditions to the subject to be treated. The modulators can be administered alone or as the active ingredient of a pharmaceutical composition. Therapeutic composition of the present invention can be combined with or used in association with other therapeutic agents. For example, a subject may be treated with an LXR agonist along with other conventional anti-diabetes drugs. Examples of such known anti-diabetes drugs include Actos (pioglitizone, Takeda, Eli Lilly), Avandia (rosiglitazone, Smithkline Beacham), Amaryl (glimepiride, Aventis), Glipizide Sulfonlyurea (Generic) or Glucotrol (Pfizer), Glucophage (metformin, Bristol Meyers Squibb), Glucovance (glyburide/metformin, Bristol Meyers Squibb), Glucotrol XL (glipizide extended release, Pfizer), Glyburide (Micronase; Upjohn, Glynase; Upjohn, Diabeta; Aventis), Glyset (miglitol, Pharmacia & Upjohn), Metaglip (glipizide+metformin; fixed combination tablet), Prandin (repaglinide, NOVO), Precose (acarbose, Bayer), Rezulin (troglitazone, Parke Davis), and Starlix (nateglinide, Novartis).

Pharmaceutical compositions of the present invention typically comprise at least one active ingredient together with one or more acceptable carriers thereof. Pharmaceutically carriers enhance or stabilize the composition, or to facilitate preparation of the composition. Pharmaceutically acceptable carriers are determined in part by the particular composition being administered (e.g., nucleic acid, protein, or modulatory compounds), as well as by the particular method used to administer the composition. They should also be both pharmaceutically and physiologically acceptable in the sense of being compatible with the other ingredients and not injurious to the subject. This carrier may take a wide variety of forms depending on the form of preparation desired for administration, e.g., oral, sublingual, rectal, nasal, or parenteral.

There are a wide variety of suitable pharmaceutically acceptable carriers to practice the present invention (see, e.g., Remington: The Science and Practice of Pharmacy, Mack Publishing Co., 20^{th} ed., 2000). Without limitation, they include syrup, water, isotonic saline solution, 5% dextrose in water or buffered sodium or ammonium acetate solution, oils, glycerin, alcohols, flavoring agents, preservatives, coloring agents starches, sugars, diluents, granulating agents, lubricants, and binders, among others. The LXR agonist can also be complexed with carrier proteins such as ovalbumin or serum albumin prior to their administration in order to enhance stability.

The pharmaceutical compositions can be prepared in various forms, such as granules, tablets, pills, suppositories, capsules, suspensions, salves, lotions and the like. The concentration of therapeutically active compound in the formulation may vary from about 0.1-100% by weight. Therapeutic formulations are peprared by any methods well known in the art of pharmacy. See, e.g., Gilman et al., eds., Goodman and Gilman's: The Pharmacological Bases of Therapeutics, 8th ed., Pergamon Press, 1990; Remington: The Science and Practice of Pharmacy, Mack Publishing Co., 20^{th} ed., 2000; Avis et al., eds., Pharmaceutical Dosage Forms: Parenteral Medications, published by Marcel Dekker, Inc., N.Y., 1993; and Lieberman et al., eds., Pharmaceutical Dosage Forms: Disperse Systems, published by Marcel Dekker, Inc., N.Y., 1990.

A pharmaceutical composition of the invention is formulated to be compatible with its intended route of administration. Examples of suitable routes of administration include parenteral, e.g., intravenous, intradermal, subcutaneous, oral (e.g., inhalation), transdermal (topical), transmucosal, and rectal administration. For parenteral administration, the LXR agonists of the present invention may be formulated in a variety of ways. Aqueous solutions of the modulators may be encapsulated in polymeric beads, nanoparticles or other injectable depot formulations known to those of skill in the art. Additionally, the compounds of the present invention may also be administered encapsulated in liposomes. The compositions, depending upon its solubility, may be present both in the aqueous layer and in the lipidic layer, or in what is generally termed a liposomic suspension. The hydrophobic layer, generally but not exclusively, comprises phospholipids such as lecithin and sphingomyelin, steroids such as cholesterol, more or less ionic surfactants such a diacetylphosphate, stearylamine, or phosphatidic acid, and/or other materials of a hydrophobic nature.

The compositions may be supplemented by other active pharmaceutical ingredients, where desired. Optional antibacterial, antiseptic, and antioxidant agents may also be present in the compositions where they will perform their ordinary functions. In some applications, the LXR agonists are prepared with carriers that will protect the compound against rapid elimination from the body, such as a controlled release formulation, including implants and microencapsulated delivery systems. A sustained release material such as glyceryl monostearate or glyceryl distearate, alone or with a wax can be included in the compositions. Biodegradable, biocompatible polymers can also be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc.

The pharmaceutical compositions can be included in a container, pack, or dispenser together with instructions for administration.

C. Dosages

Subjects suffering from diabetes or related disorders are typically treated with pharmaceutical compositions of the present invention for a continued period of time (e.g., at least 10 days, 14 days, 30 days, 60 days, 90 days, or longer). The pharmaceutical compositions comprise a pharmaceutically effective amount or prophylactically effective amount of an LXR agonist. The term “therapeutically effective amount” is intended to mean that amount of a drug or pharmaceutical agent that will elicit the biological or medical response of a tissue, a system, animal or human that is being sought by a researcher, veterinarian, medical doctor or other clinician. The term “prophylactically effective amount” is intended to mean that amount of a pharmaceutical drug that will prevent or reduce the risk of occurrence of the biological or medical event that is sought to be prevented in a tissue, a system, animal or human.

A suitable therapeutic dose can be determined by any of the well-known methods such as clinical studies on mammalian species to determine maximum tolerable dose and on normal human subjects to determine safe dosage. Particularly, the dosage amount of an LXR ligand that a subject receives can be selected so as to achieve the desired up-regulation of glut4 expression; the dosage a subject receives may also be titrated over time in order to reach a target Glut4 level. Toxicity and therapeutic efficacy of LXR agonists can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. LXR agonists that exhibit large therapeutic indices are preferred. While LXR agonists that exhibit toxic side effects may be used, care should be taken to design a delivery system that targets such LXR agonists to the site of affected tissue in order to minimize potential damage to uninfected cells and, thereby, reduce side effects.

The data obtained from the cell culture assays and animal studies can be used in formulating a range of dosages for use in humans. The dosage of such compounds lies preferably within a range of circulating concentrations that include the ED₅₀ with little or no toxicity. The dosage may vary within this range depending upon the dosage form employed and the route of administration utilized. For any LXR agonist used in the method of the invention, the therapeutically effective dose can be estimated initially from cell culture assays. A dose may be formulated in animal models to achieve a circulating plasma concentration range that includes the IC₅₀ (i.e., the concentration of the test LXR agonists which achieves a half-maximal inhibition of symptoms) as determined in cell culture. Such information can be used to more accurately determine useful doses in humans. Levels in plasma may be measured, for example, by high performance liquid chromatography.

In general, except under certain circumstances when higher dosages may be required, the preferred dosage of an LXR agonist usually lies within the range of from about 0.001 to about 1000 mg, more usually from about 0.01 to about 500 mg per day. The preferred dosage and mode of administration of an LXR agonist can vary for different subjects, depending upon factors that can be individually reviewed by the treating physician, such as the condition or conditions to be treated, the choice of composition to be administered, including the particular LXR agonist, the age, weight, and response of the individual subject, the severity of the subject's symptoms, and the chosen route of administration. As a general rule, the quantity of an LXR agonist administered is the smallest dosage that effectively and reliably prevents or minimizes the conditions of the subjects. Therefore, the above dosage ranges are intended to provide general guidance and support for the teachings herein, but are not intended to limit the scope of the invention.

In some applications, a first LXR agonist is used in combination with a second LXR agonist or a known anti-diabetes drug in order to achieve therapeutic effects that cannot be achieved when just one LXR agonist is used individually.

D. Administration of Polynucleotides Encoding LXR Agonists or LXR

In some methods of the present invention, polynucleotides encoding LXR agonists of the present invention are transfected into cells for therapeutic purposes in vitro and in vivo. These polynucleotides can be inserted into any of a number of well-known vectors for the transfection of target cells and organisms as described below. The polynucleotides are transfected into cells, ex vivo or in vivo, through the interaction of the vector and the target cell. The compositions are administered to a subject in an amount sufficient to elicit a therapeutic response in the subject.

In some related embodiments, rather than administering polynucleotides encoding an LXR agonist, a polynucleotide encoding the LXR receptor can be transfected into cells or administered to a subject for therapeutic purposes. The subject can be further administered an LXR agonist (e.g., a small molecule LXR agonist) as described above. Expression of the exogenous LXR receptors and administration of the LXR agonist could stimulate LXR mediated pathway, including regulation of glucose metabolism.

Such gene therapy procedures have been used to correct acquired and inherited genetic defects, cancer, and viral infection in a number of contexts. The ability to express artificial genes in humans facilitates the prevention and/or cure of many important human diseases, including many diseases which are not amenable to treatment by other therapies (for a review of gene therapy procedures, see Anderson, Science 256:808-813 (1992); Nabel & Felgner, TIBTECH 11:211-217 (1993); Mitani & Caskey, TIBTECH 11:162-166 (1993); Mulligan, Science 926-932 (1993); Dillon, TIBTECH 11:167-175 (1993); Miller, Nature 357:455-460 (1992); Van Brunt, Biotechnology 6(10):1149-1154 (1998); Vigne, Restorative Neurology and Neuroscience 8:35-36 (1995); Kremer & Perricaudet, British Medical Bulletin 51(1):31-44 (1995); Haddada et al., in Current Topics in Microbiology and Immunology (Doerfler & Böhhm eds., 1995); and Yu et al., Gene Therapy 1:13-26 (1994)).

Delivery of the gene or genetic material into the cell is the first step in gene therapy treatment of disease. A large number of delivery methods are well known to those of skill in the art. Preferably, the polynucleotides are administered for in vivo or ex vivo gene therapy uses. Non-viral vector delivery systems include DNA plasmids, naked nucleic acid, and nucleic acid complexed with a delivery vehicle such as a liposome. Viral vector delivery systems include DNA and RNA viruses, which have either episomal or integrated genomes after delivery to the cell.

Methods of non-viral delivery of nucleic acids include lipofection, microinjection, biolistics, virosomes, liposomes, immunoliposomes, polycation or lipid:nucleic acid conjugates, naked DNA, artificial virions, and agent-enhanced uptake of DNA. Lipofection is described in, e.g., U.S. Pat. No. 5,049,386, U.S. Pat. No. 4,946,787; and U.S. Pat. No. 4,897,355, and lipofection reagents are sold commercially (e.g., Transfectam™ and Lipofectin™). Cationic and neutral lipids that are suitable for efficient receptor-recognition lipofection of polynucleotides include those described in WO 91/17424 and WO 91/16024. Delivery can be directed to cells (ex vivo administration) or target tissues (in vivo administration).

The preparation of lipid:nucleic acid complexes, including targeted liposomes such as immunolipid complexes, is well known to one of skill in the art (see, e.g., Crystal, Science 270:404-410 (1995); Blaese et al., Cancer Gene Ther. 2:291-297 (1995); Behr et al., Bioconjugate Chem. 5:382-389 (1994); Remy et al., Bioconjugate Chem. 5:647-654 (1994); Gao et al., Gene Therapy 2:710-722 (1995); Ahmad et al., Cancer Res. 52:4817-4820 (1992); U.S. Pat. Nos. 4,186,183, 4,217,344, 4,235,871, 4,261,975, 4,485,054, 4,501,728, 4,774,085, 4,837,028, and 4,946,787).

Gene therapy vectors can be delivered in vivo by administration to an individual subject, typically by systemic administration (e.g., intravenous, intraperitoneal, intramuscular, subdermal, or intracranial infusion) or topical application, as described below. Alternatively, vectors can be delivered to cells ex vivo, such as cells explanted from an individual subject (e.g., lymphocytes, bone marrow aspirates, tissue biopsy) or universal donor hematopoietic stem cells, followed by reimplantation of the cells into a subject, usually after selection for cells which have incorporated the vector.

Ex vivo cell transfection for diagnostics, research, or for gene therapy (e.g., via re-infusion of the transfected cells into the host organism) is well known to those of skill in the art. In a preferred embodiment, cells are isolated from the subject organism, transfected with a nucleic acid (gene or cDNA), and re-infused back into the subject organism (e.g., subject). Various cell types suitable for ex vivo transfection are well known to those of skill in the art (see, e.g., Freshney et al., Culture of Animal Cells, A Manual of Basic Technique (3rd ed. 1994)) and the references cited therein for a discussion of how to isolate and culture cells from subjects).

Vectors (e.g., retroviruses, adenoviruses, liposomes, etc.) containing therapeutic nucleic acids can be also administered directly to the organism for transduction of cells in vivo. Alternatively, naked DNA can be administered. Administration is by any of the routes normally used for introducing a molecule into ultimate contact with blood or tissue cells. Suitable methods of administering such nucleic acids are available and well known to those of skill in the art, and, although more than one route can be used to administer a particular composition, a particular route can often provide a more immediate and more effective reaction than another route.

The use of RNA or DNA viral based systems for the delivery of nucleic acids take advantage of highly evolved processes for targeting a virus to specific cells in the body and trafficking the viral payload to the nucleus. Viral vectors can be administered directly to subjects (in vivo) or they can be used to treat cells in vitro and the modified cells are administered to subjects (ex vivo). Conventional viral based systems for the delivery of nucleic acids could include retroviral, lentivirus, adenoviral, adeno-associated and herpes simplex virus vectors for gene transfer. Viral vectors are currently the most efficient and versatile method of gene transfer in target cells and tissues. Integration in the host genome is possible with the retrovirus, lentivirus, and adeno-associated virus gene transfer methods, often resulting in long term expression of the inserted transgene. Additionally, high transduction efficiencies have been observed in many different cell types and target tissues.

The tropism of a retrovirus can be altered by incorporating foreign envelope proteins, expanding the potential target population of target cells. Lentiviral vectors are retroviral vector that are able to transduce or infect non-dividing cells and typically produce high viral titers. Selection of a retroviral gene transfer system would therefore depend on the target tissue. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Widely used retroviral vectors include those based upon murine leukemia virus (MuLV), gibbon ape leukemia virus (GaLV), simian immunodeficiency virus (SUV), human immunodeficiency virus (HIV), and combinations thereof (see, e.g., Buchscher et al., J. Virol. 66:2731-2739 (1992); Johann et al., J. Virol. 66:1635-1640 (1992); Sommerfelt et al., Virol. 176:58-59 (1990); Wilson et al., J. Virol. 63:2374-2378 (1989); Miller et al., J. Virol. 65:2220-2224 (1991); PCT/US94/05700).

In particular, a number of viral vector approaches are available for gene transfer in clinical trials, with retroviral vectors by far the most frequently used system. All of these viral vectors utilize approaches that involve complementation of defective vectors by genes inserted into helper cell lines to generate the transducing agent. For example, pLASN and MFG-S are examples are retroviral vectors that have been used in clinical trials (Dunbar et al., Blood 85:3048-305 (1995); Kohn et al., Nat. Med. 1:1017-102 (1995); Malech et al., Proc. Natl. Acad. Sci. U.S.A. 94:22 12133-12138 (1997)). PA317/pLASN was the first therapeutic vector used in a gene therapy trial. (Blaese et al., Science 270:475-480 (1995)). Transduction efficiencies of 50% or greater have been observed for MFG-S packaged vectors (Ellem et al., Immunol Immunother. 44(1):10-20 (1997); Dranoff et al., Hum. Gene Ther. 1:111-2 (1997)).

In many gene therapy applications, it is desirable that the gene therapy vector be delivered with a high degree of specificity to a particular tissue type. A viral vector is typically modified to have specificity for a given cell type by expressing a ligand as a fusion protein with a viral coat protein on the viruses outer surface. The ligand is chosen to have affinity for a receptor known to be present on the cell type of interest. For example, Han et al., Proc. Natl. Acad. Sci. U.S.A. 92:9747-9751 (1995), reported that Moloney murine leukemia virus can be modified to express human heregulin fused to gp70, and the recombinant virus infects certain human breast cancer cells expressing human epidermal growth factor receptor. This principle can be extended to other pairs of virus expressing a ligand fusion protein and target cell expressing a receptor. For example, filamentous phage can be engineered to display antibody fragments (e.g., FAB or Fv) having specific binding affinity for virtually any chosen cellular receptor. Although the above description applies primarily to viral vectors, the same principles can be applied to nonviral vectors. Such vectors can be engineered to contain specific uptake sequences thought to favor uptake by specific target cells.

### EXAMPLES

The following examples are offered to illustrate, but not to limit the present invention.

### Example 1

### LXR Agonists Modulate Expressions of Glucose Metabolism-Related Genes

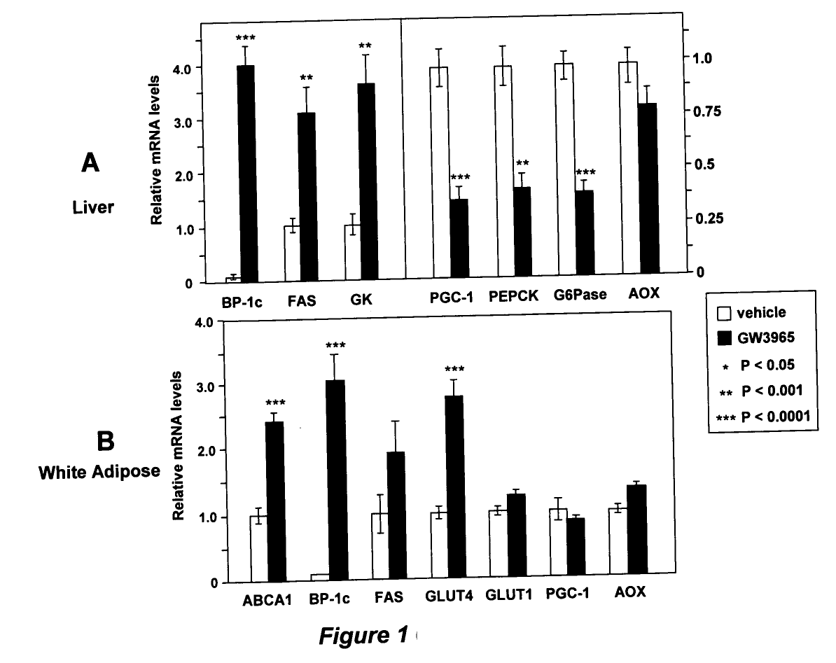

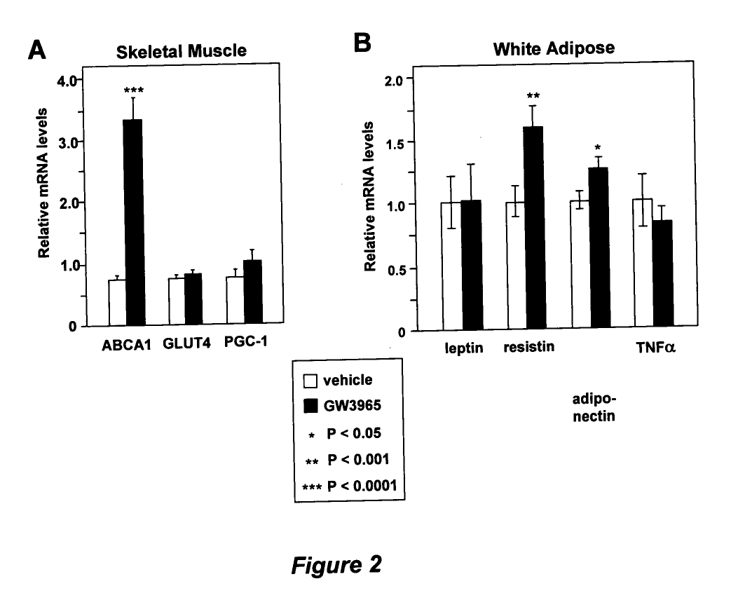

This Example describes coordinated regulation of genes involved in glucose metabolism by LXR agonist in vivo. 10 week old female C57/B16 mice (n=9 per group) were gavaged daily with GW3965 (20 mg/kg/day) or vehicle. At the end of the treatment period, mice were fasted for 12 hours, sacrificed and total RNA was isolated. Gene expression for individual animals was determined by real time quantitative PCR assays. The results are presented in FIGS. 1 and 2 as average expression for each group +/−standard deviation.

FIG. 1A shows that LXR ligand GW3965 induces glucokinase expression and represses genes involved in gluconeogenesis in liver. The LXR ligand also induces GLUT4 expression in white adipose tissue (FIG. 1B). FIG. 2A shows that LXR ligands regulate ABCA1, but do not alter GLUT4 or PGC-1 expression in skeletal muscle. Effects of LXR ligands on expression of adipocyte signaling molecules are shown in FIG. 2B.

### Example 2

### The Modulatory Activities of LXR Agonists is Dependent on LXR

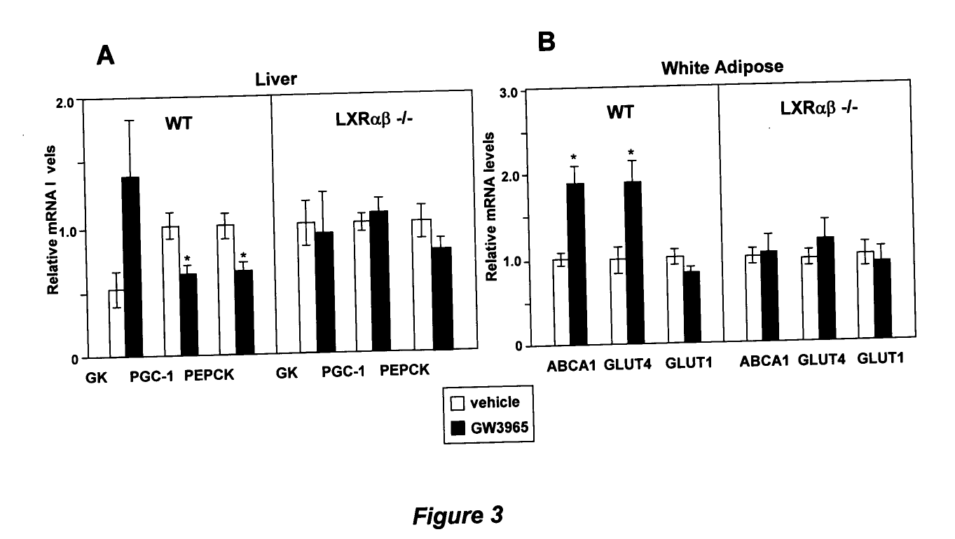

This Example describes that the effects of LXR agonists on expression of genes involved in glucose metabolism is dependent on LXR expression. LXR null mice or wild-type controls on a mixed background (n=4 per group) were gavaged daily with GW3965 (20 mg/kg/day) or vehicle. At the end of the treatment period, mice were fasted for 12 hours, sacrificed and total RNA was isolated. Gene expression for was determined by real time quantitative PCR assays. FIG. 3A shows that regulation of PGC-1 and PEPCK expression by GW3965 is abolished in livers of LXR null mice. Similarly, FIG. 3B demonstrates that regulation of GLUT4 expression is abolished in adipose tissue of LXR null mice.

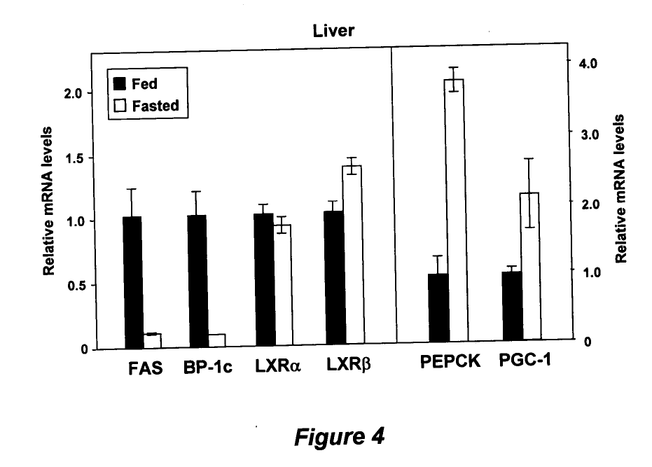

To determine whether expression of LXRs is altered by fasting, C57/B16 mice (n=4 per group) were fasted for 12 hours, sacrificed and total RNA was isolated. Gene expression was determined by real time quantitative PCR assays. Relative mRNA expression levels are presented in FIG. 4. The results indicate that fasting does not alter expression of the receptor.

### Example 3

### Induction of Glut4 Expression by LXR Agonists

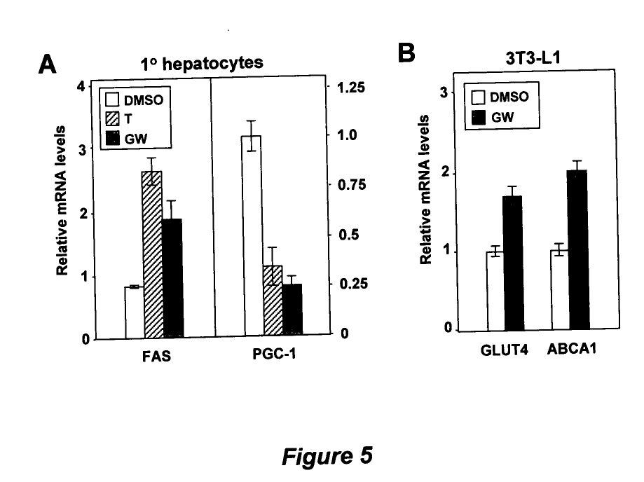

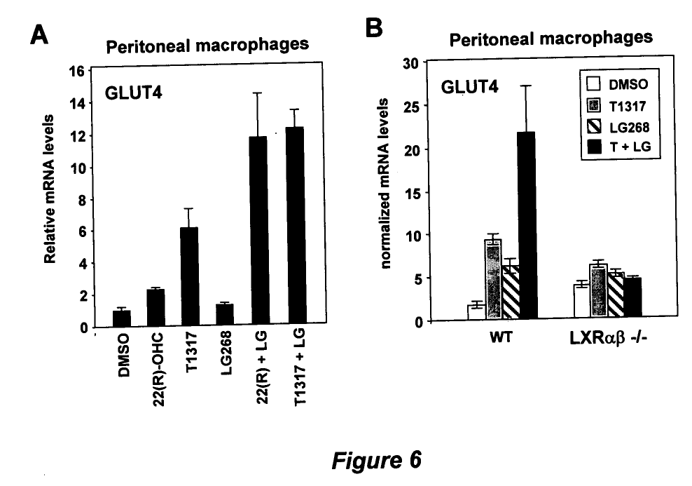

This Example shows that LXR agonists regulate GLUT4 and PGC-1 expressions in a cell autonomous manner. Cells were treated with vehicle or 1 μM T1317, 1 μM GW3965, 2 μM 22(R)-hydroxycholesterol or 50 nM LG268 for 24 hours as indicated. mRNA expression was determined by real time quantitative PCR assays. The results indicate that LXR ligands repress PGC-1 expression in primary human hepatocytes (FIG. 5A) and induce GLUT4 expression in 3T3-L1 adipocytes (FIG. 5B). FIG. 6A shows modulation of Glut4 expression in macrophages by synthetic and oxysterol LXR ligands. Regulation of Glut4 expression by the LXR ligands is abolished in cells from LXR null mice.

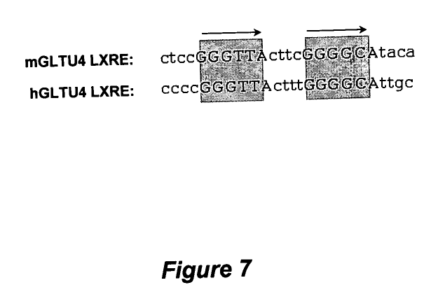

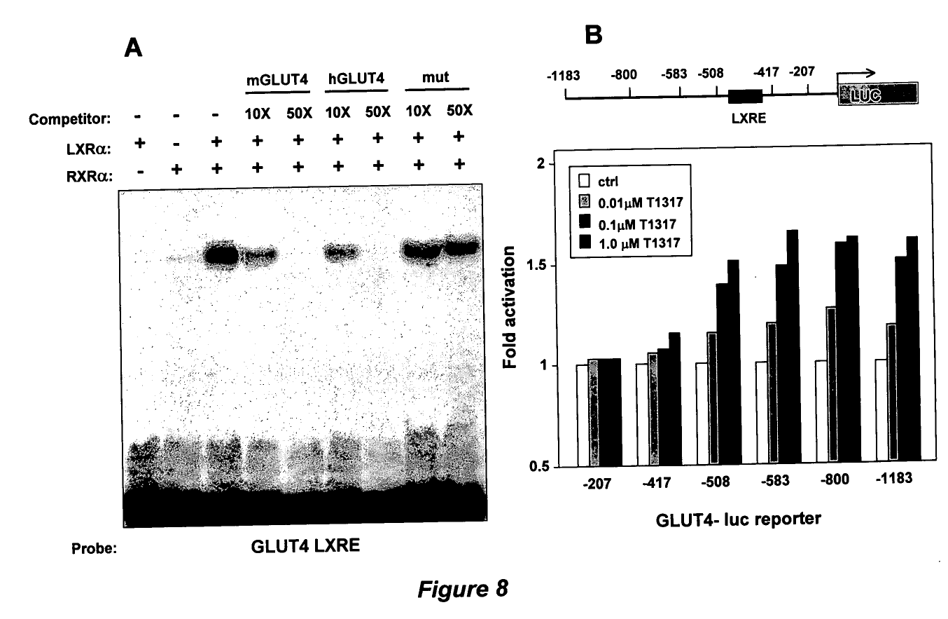

This Example also demonstrates that the GLUT4 promoter is a direct target for regulation by LXR. FIG. 7 shows a conserved DR-4 hormone response element in the mouse Glut4 promoter (SEQ ID NO: 1) and human Glut4 promoter (SEQ ID NO: 2). To examine whether the GLUT4 promoter is a direct target for regulation by LXR/RXR heterodimers, electromobility shift assays were performed using in vitro translated proteins and radiolabeled mGLUT4 oligonucleotide (FIG. 8A). The results indicate a functional LXR binding site in the GLUT4 promoter. To further examine whether LXR ligands activate the human GLUT4 promoter, clonal populations of 3T3-L1 cells carrying stably-integrated luciferase reporters under the control of various truncations of the human GLUT4 promoter were differentiated into adipocytes. On day 8 of differentiation, cells were incubated in serum-free media with the indicated concentrations of T1317. Luciferase activity was measured 24 hours later. As shown in FIG. 8B, the results indicate that the human GLUT4 promoter is a direct target for regulation by the LXR agonist. Sequence alignment of LXREs in the mouse and human GLUT4 promoters is also shown in FIG. 7.

### Example 4

### LXR Agonists Enhance Glucose Uptake and Glucose Tolerance

This Example demonstrates that LXR agonists enhance glucose uptake in adipocytes. 3T3-L1 adipocytes at day 10 of differentiation were incubated for 24 hours with serum-free media supplemented with the indicated concentrations of T1317. The following day, basal glucose uptake (in the absence of insulin) of treated cells was measured in 96-well CytostarT plates using 14 C-labeled 2-DOG. Insulin (1 μg/ml) was used as positive control.

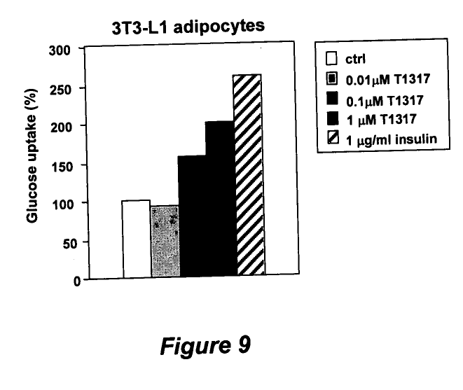

The results as shown in FIG. 9 indicate that the LXR agonist promotes glucose uptake in the adipocytes.

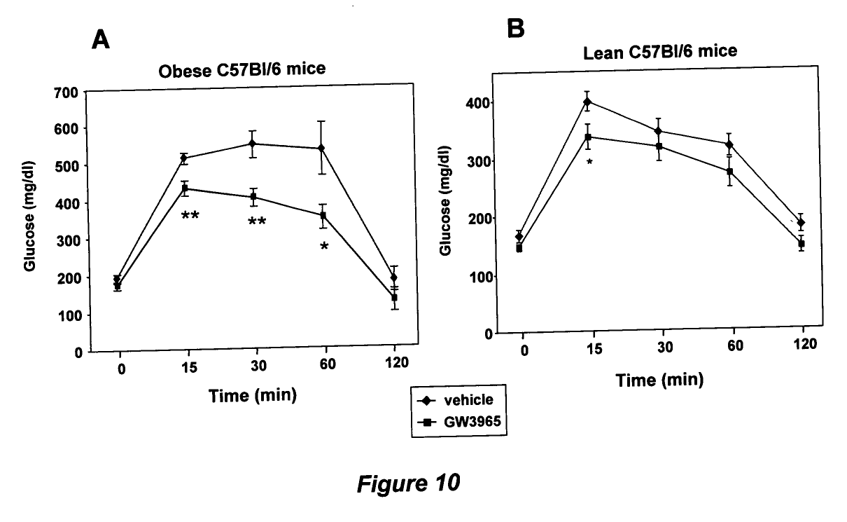

To further examine effects of LXR agonists on glucose tolerance, C57B16 mice were fed a high fat diet for three months (Clinton/Cybulsky rodent diet; 40% kcal from fat, devoid of cholesterol) to induce obesity. After 3 months, mice were treated for one week with vehicle or 20 mg/kg/day GW3965. Glucose tolerance tests were performed by intraperitoneal injection of glucose (2 g/kg body weight) after 8 hours of fasting (FIG. 1A). In addition, effect of GW3965 on glucose tolerance in lean C57B1/6 mice was also examined (FIG. 10B). The lean C57B1/6 mice maintained on normal chow diet and similarly treated as indicated above. The results indicate that LXR ligands improve glucose tolerance and insulin resistance in diet-induced obesity.

### Example 5

### Synergistic Effects Between LXR Agonists and Anti-Diabetic Drugs

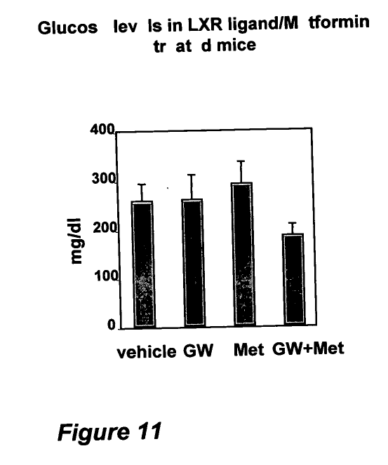

This Example describes treatment of mice with a combination of an LXR agonist and a known anti-diabetic drug. Obese, insulin-resistant ob/ob mice were treated with an LXR ligand, GW3965, at 20 mg/kg/day and/or metformin at 300 mg/kg/day for 3 months. Fasting plasma glucose levels were then determined. The results indicate that mice treated with the combination of LXR ligand and metformin had circulating glucose level that is significantly lower than that in mice treated with either compound alone. As shown in FIG. 11, under the given experimental conditions, plasma glucose levels in mice treated with the LXR agonist or metformin alone did not show significant difference from that of control (mice treated with vehicle). By contrast, when mice were treated with a combination of GW3965 and metformin, their plasma glucose level was significantly reduced as compared to that in the control mice. These data suggest that there could be a synergistic effect between LXR agonists and certain known anti-diabetic compounds in treating diabetes.

It is understood that the examples and embodiments described herein are for illustrative purposes only and that various modifications or changes in light thereof will be suggested to persons skilled in the art and are to be included within the spirit and purview of this application and scope of the appended claims. Although any methods and materials similar or equivalent to those described herein can be used in the practice or testing of the present invention, the preferred methods and materials are described.

All publications, GenBank sequences, patents and patent applications cited herein are hereby expressly incorporated by reference in their entirety and for all purposes as if each is individually so denoted.

1. A method for enhancing glut4 expression in a cell, the method comprising (i) providing a cell expressing glut4 gene; and (ii) contacting the cell with an LXR agonist; thereby enhancing glut4 expression level in the cell.

2. The method of claim 1, further comprising measuring glut4 expression level in the cell before and/or after administering the LXR agonist.

3. The method of claim 1, wherein the cell is an adipose cell.

4. The method of claim 1, wherein the LXR agonist is an LXRβ agonist.

5. The method of claim 4, where the LXRβ agonist is selected from the group consisting of GW3965, F3MethylAA, and T0901317.

6. The method of claim 1, wherein the cell is present in a subject.

7. The method of claim 6, wherein the subject is suffering from type II diabetes.

8. The method of claim 7, wherein the subject is administered with a pharmaceutical composition comprising an effective amount of the LXR agonist.

9. The method of claim 8, wherein the subject is administered simultaneously with a known anti-diabetic drug to the subject.

10. The method of claim 9, wherein the known anti-diabetic drug is metformin.

11. The method of claim 1, wherein the LXR agonist is identified by screening a library of test agents.

12. A method for ameliorating type II diabetes in a subject, the method comprising administering to the subject an effective amount of an LXR agonist; thereby ameliorating type II diabetes in the subject.

13. The method of claim 12, further comprising measuring circulating glucose level in the subject before and/or after administering the LXR agonist.

14. The method of claim 12, wherein the LXR agonist is identified by screening a library of test agents.

15. The method of claim 12, wherein the LXR agonist is an LXRβ agonist.

16. The method of claim 12, wherein the LXR agonist is administered to the subject at least daily for at least 14 days.

17. The method of claim 12, wherein the LXR agonist is administered simultaneously with a known anti-diabetic drug to the subject.

18. The method of claim 17, wherein the known anti-diabetic drug is metformin.

19. A method for enhancing insulin sensitivity and glucose uptake by a cell in a subject, the method comprising administering to the subject an effective amount of an LXR agonist; thereby enhancing insulin sensitivity and glucose uptake by the cell.

20. The method of claim 19, wherein the subject is suffering from type II diabetes.

21. The method of claim 19, wherein the LXR agonist is an LXRβ agonist.

22. The method of claim 21, where the LXRβ agonist is selected from the group consisting of GW3965, F3MethylAA, and T0901317.

23. The method of claim 19, wherein the cell is an adipose cell.

24. The method of claim 19, wherein the LXR agonist is administered simultaneously with a known anti-diabetic drug to the subject.

25. The method of claim 24, wherein the known anti-diabetic drug is metformin.

26. A method for reducing gluconeogenesis in a subject, the method comprising (i) screening test agents to identify an LXR agonist, and (ii) administering to the subject an effective amount of the LXR agonist; thereby reducing gluconeogenesis in the subject.

27. The method of claim 26, wherein the subject is suffering from type II diabetes.

28. The method of claim 26, wherein the LXR agonist is an LXRβ agonist.

29. The method of claim 28, where the LXRβ agonist is GW3965.
